(19) United States
(12) Patent Application Publication
(10) Pub. No.: US 2025/0375119 A1
(43) Pub. Date: Dec. 11, 2025

(54) CONDITION ANALYZER AND CONDITION ANALYSIS METHOD

(71) Applicant: NIPPON TELEGRAPH AND TELEPHONE CORPORATION, Tokyo (JP)

(72) Inventors: Takayuki OGASAWARA, Musashino-shi (JP); Toichiro GOTO, Musashino-shi (JP); Masumi YAMAGUCHI, Musashino-shi (JP); Kentaro TANAKA, Musashino-shi (JP); Shingo TSUKADA, Musashino-shi (JP)

(73) Assignee: NIPPON TELEGRAPH AND TELEPHONE CORPORATION, Tokyo (JP)

(21) Appl. No.: 18/876,945

(22) PCT Filed: Jun. 29, 2022
(86) PCT No.: PCT/JP2022/026054

§ 371 Date: Dec. 19, 2024

(51) Int. Cl. A61B 5/0255; A61B 5/00; A61B 5/11

(52) U.S. Cl. CPC A61B 5/0255; A61B 5/11; A61B 5/742

(57) ABSTRACT

An aspect of the present invention is a condition analysis device including an acquisition unit that acquires rank information obtained by ranking a degree of self-support or magnitude of disorder of a patient and biometric information of the patient, a derivation unit that derives, for each rank, range information indicating a range in which the biometric information is distributed, and a superimposition display unit that displays the range information as an image for each rank, and superimposes and displays a predetermined image on the image at a position corresponding to the biometric information of one patient in a range indicated by the image.

### TECHNICAL FIELD

The present invention relates to a condition analysis device and a condition analysis method.

### BACKGROUND ART

In rehabilitation medicine, functional independence measure (FIM) and stroke impairment assessment set (SIAS) are used as measures for evaluating a patient when the patient suffers from disorder due to paralysis associated with a cerebrovascular disease or the like. These are acquisition rank information obtained by a medical practitioner scoring the degree of self-support of the patient and the magnitude of the disorder using a predetermined measure. Using a wearable device facilitates acquiring the amount of activity in daily life. Briskness in life can be known by such an amount of activity being acquired.

### CITATION LIST

### Non Patent Document

Non Patent Document 1: Technical Information Institute Co., Ltd., Biometric Information Sensing and Application to Human State Estimation, 2020.
Non Patent Document 2: Outlier Exclusion Method https://toukeigaku-jouhou.info/2018/04/25/determine-outlier/Non
Patent Document 3: Missing Data Processing by Multiple Substitution Method https://qiita.com/saltcooky/items/2e39c4bc099d20f20d59

### SUMMARY OF INVENTION

### Technical Problem

One of the purposes of rehabilitation is to bring a patient back to daily life, and recovery of the amount of activity to a healthy amount is an important point of view. However, there has not been proposed an effective method for visualizing the amount of activity known from acquisition rank information and biometric data and quantifying the relevance. Therefore, it has been difficult to provide suitable assistance regarding rehabilitation, such as assistance for creation of a rehabilitation treatment plan and assistance for improving motivation of a patient for rehabilitation training.

In view of the above circumstances, an object of the present invention is to provide a technology capable of providing suitable assistance regarding rehabilitation.

### Solution to Problem

An aspect of the present invention is a condition analysis device including an acquisition unit that acquires rank information obtained by ranking a degree of self-support or magnitude of disorder of a patient and biometric statistical information of the patient, a derivation unit that derives, for each rank, range information indicating a range in which the biometric statistical information is distributed, and a superimposition display unit that displays the range information as an image for each rank, and superimposes and displays a predetermined image on the image at a position corresponding to the biometric statistical information of one patient in a range indicated by the image.

An aspect of the present invention is a condition analysis method including an acquisition step of acquiring rank information obtained by ranking a degree of self-support or magnitude of disorder of a patient and biometric statistical information of the patient, a derivation step of deriving, for each rank, range information indicating a range in which the biometric statistical information is distributed, and a superimposition display step of displaying the range information as an image for each rank, and superimposing and displaying a predetermined image on the image at a position corresponding to the biometric statistical information of one patient in a range indicated by the image.

### Advantageous Effects of Invention

According to the present invention, suitable assistance regarding rehabilitation can be provided.

### BRIEF DESCRIPTION OF DRAWINGS

FIG. 1 A functional block diagram illustrating a functional configuration of a condition analysis device according to a first embodiment.

FIG. 2 A diagram illustrating a specific example of a patient database.

FIG. 3 A diagram illustrating a specific example of a statistical database.

FIG. 4 A diagram illustrating an example of a graph.

FIG. 5 A flowchart illustrating a flow of processing of the condition analysis device.

FIG. 6 A functional block diagram illustrating a functional configuration of a condition analysis device according to a second embodiment.

FIG. 7 A diagram illustrating an example using quartile values.

FIG. 8 A diagram illustrating a graph after exclusion.

FIG. 9 A functional block diagram illustrating a functional configuration of a condition analysis device according to a third embodiment.

FIG. 10 A diagram illustrating a display example by a tendency display unit.

FIG. 11 A functional block diagram illustrating a functional configuration of a condition analysis device according to a fourth embodiment.

FIG. 12 A diagram illustrating an example of a graph on which predicted rank statistical information and biometric statistical information are superimposed.

FIG. 13 A diagram illustrating correlation coefficients of predicted values.

FIG. 14 A functional block diagram illustrating a functional configuration of a condition analysis device according to a fifth embodiment.

### DESCRIPTION OF EMBODIMENTS

### First Embodiment

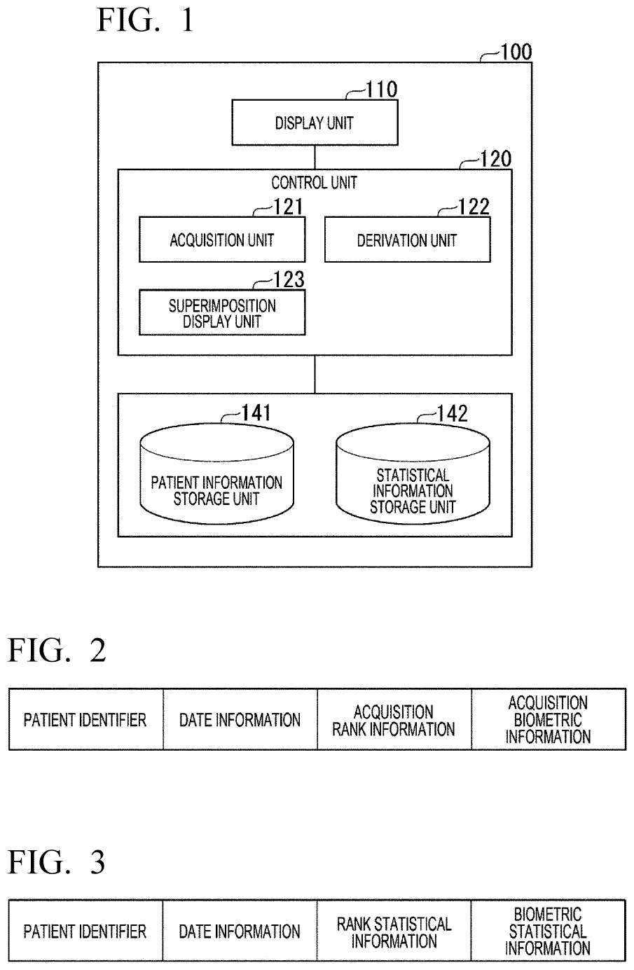

FIG. 1 is a functional block diagram illustrating a functional configuration of a condition analysis device 100. The condition analysis device 100 includes a central processing unit (CPU), a memory, an auxiliary storage device, and the like connected by a bus, and functions as a device including a display unit 110, a patient information storage unit 141, a statistical information storage unit 142, and a control unit 120 by executing a condition analysis program. Note that all or some of functions of the display unit 110, the patient information storage unit 141, and the control unit 120 may be implemented using hardware such as an application specific integrated circuit (ASIC), a programmable logic device (PLD), or a field programmable gate array (FPGA). The condition analysis program may be recorded on a computer-readable recording medium. The computer-readable recording medium is, for example, a portable medium such as a flexible disk, a magneto-optical disc, a ROM, or a CD-ROM or a storage device such as a hard disk built in a computer system. The condition analysis program may be transmitted via an electrical communication line.

The display unit 110 is a display device that displays various types of information by liquid crystal, organic electro luminescence (EL), or the like.

The patient information storage unit 141 is configured using a storage device such as a magnetic hard disk device or a semiconductor storage device. The patient information storage unit 141 stores a patient database. FIG. 2 is a diagram illustrating a specific example of the patient database. The patient database includes a patient identifier, date information, acquisition rank information, and acquisition biometric information.

The patient identifier is an identifier for uniquely identifying a patient. The date information indicates a date when the acquisition rank information and the acquisition biometric information are acquired. The acquisition rank information is information obtained by ranking the degree of self-support of a patient or the magnitude of disorder. The ranking of the degree of self-support of a patient is performed by, for example, the functional independence measure (FIM). The ranking of the magnitude of disorder is performed by the stroke impairment assessment set (SIAS). For example, in a case of using the FIM, the acquisition rank information indicates any one of seven ranks of a rank 1 to a rank 7 for each item of 13 exercise items. The acquisition biometric information is information determined on the basis of a heart rate or body motion. For example, the acquisition biometric information is a heart rate, body motion, or exercise intensity.

The statistical information storage unit 142 is configured using a storage device such as a magnetic hard disk device or a semiconductor storage device. The statistical information storage unit 142 stores a statistical database. FIG. 3 is a diagram illustrating a specific example of the statistical database. The statistical database includes a patient identifier, date information, rank statistical information, and biometric statistical information. As described above, the patient identifier is an identifier for uniquely identifying a patient. The date information indicates a date when the rank statistical information and the biometric statistical information are acquired.

In the present embodiment, the rank statistical information indicates a value obtained by rounding down decimal places of an average value per item obtained by dividing the total score (91 points) of the FIM exercise items (13 items) by 13 to an integer. Since 91 points are divided by 13, the acquisition rank information indicates one of the ranks 1 to 7. The biometric statistical information indicates the average or standard deviation of the heart rate, the body motion, or the exercise intensity described above. The biometric statistical information is, for example, an average heart rate per minute. In the present embodiment, the rank statistical information will be described as an example of “rank information”. The biometric statistical information will be described as an example of “biometric information”.

The control unit 120 in FIG. 1 controls operation of each unit of the condition analysis device 100. The control unit 120 is implemented by, for example, a device including a processor such as a CPU and a RAM. The control unit 120 functions as an acquisition unit 121, a derivation unit 122, and a superimposition display unit 123 by executing the condition analysis program.

The acquisition unit 121 acquires the acquisition rank information and the acquisition biometric information. Examples of the acquisition method include a method of acquiring information input by a user in a text form and a method of acquiring information detected by another device such as a wearable device. The acquisition unit 121 acquires the rank statistical information described above from the acquired acquisition rank information. The acquisition unit 121 acquires the biometric statistical information described above from the acquired acquisition biometric information.

The derivation unit 122 derives range information indicating a range in which the biometric statistical information is distributed for each rank. The superimposition display unit 123 displays the range information for each rank as an image (graph), and superimposes and displays a predetermined image on the graph at a position corresponding to the biometric statistical information of one patient in the range indicated by the graph. In the present embodiment, a graph is exemplified as an example of the image, but the present invention is not limited thereto, and any image may be used as long as the image can indicate a range.

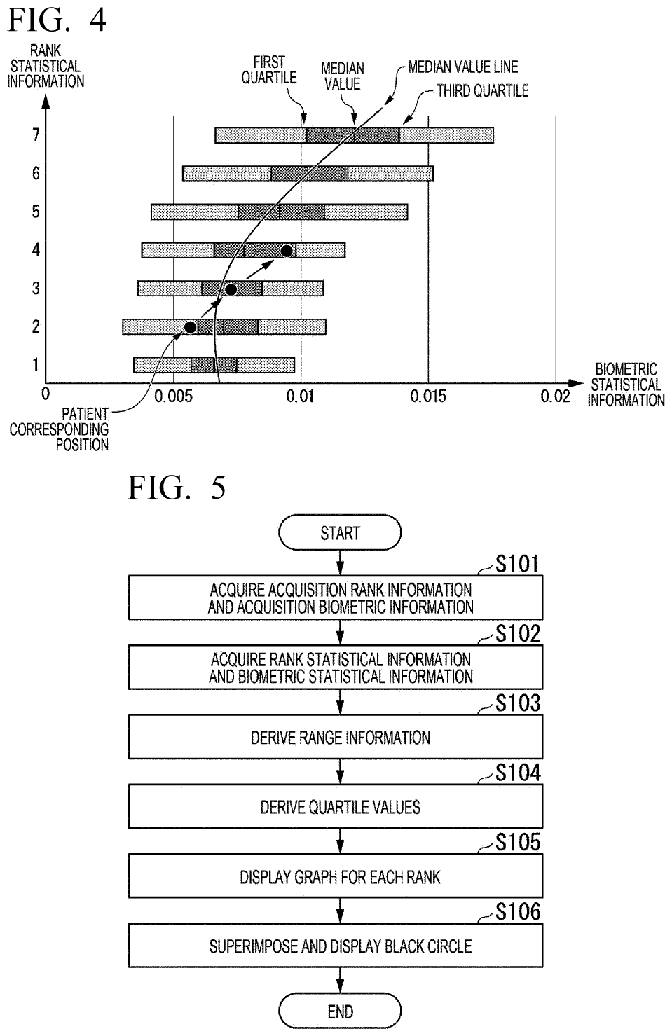

FIG. 4 is a diagram illustrating an example of the graph. The vertical axis of the graph illustrated in FIG. 4 indicates the rank statistical information, and the horizontal axis indicates the range in which the biometric statistical information is distributed. The bar graph indicates a range in which the biometric statistical information exists. The derivation unit 122 derives quartile values (first quartile, median value, third quartile) as the range information in addition to the upper limit and the lower limit of the biometric statistical information for each rank. In a case where the biometric statistical information is smaller than the median value, it indicates that the biometric statistical information is smaller than biometric information assumed from the rank. In a case where the biometric statistical information is larger than the median value, it indicates that the biometric statistical information is larger than biometric information assumed from the rank.

The superimposition display unit 123 superimposes and displays a predetermined image (black circle in FIG. 4) on the graph at a position corresponding to the biometric statistical information of one patient in the range indicated by the graph. Here, the “one patient” is a patient designated in advance by an operator of the condition analysis device 100 in the condition analysis device 100, and a patient identifier indicating this patient is stored in a nonvolatile memory (not illustrated) or the like.

Note that although a plurality of black circles and arrows is illustrated in FIG. 4, this indicates an example in which a history of a black circle is also displayed, for example. In a case of FIG. 4, it is indicated that the biometric information increases as the rank increases. That is, improvement is indicated. A black circle is used as the predetermined image, but the present invention is not limited thereto. The predetermined image may be any image as long as it is an image indicating that the predetermined image is an image corresponding to the biometric statistical information of one patient.

As illustrated in FIG. 4, the relevance between the rank statistical information and the biometric statistical information can be quantitatively visualized and the status of one patient can be indicated by the predetermined image being superimposed and displayed on the graph at a position in the range indicated by the graph corresponding to the biometric statistical information of the patient. Since such quantification cannot be performed conventionally, a patient often performs rehabilitation with an uneasy feeling without knowing well, whereas in the present embodiment, the uneasy feeling and the like can be eliminated since the patient can know his/her status, which contributes to suitable assistance regarding rehabilitation. Furthermore, displaying a history can contribute to improvement of motivation of a patient for rehabilitation training.

FIG. 5 is a flowchart illustrating a flow of processing of the condition analysis device 100. The acquisition unit 121 acquires acquisition rank information and acquisition biometric information (step S101). The acquisition unit 121 acquires rank statistical information and biometric statistical information from the acquired acquisition rank information and acquisition biometric information (step S102). The derivation unit 122 derives range information (step S103), and further derives quartile values (step S104).

The superimposition display unit 123 displays the range information for each rank as a graph (step S105), and superimposes and displays a black circle on the graph at a position corresponding to the biometric statistical information of one patient in the range indicated by the graph (step S106).

According to the present embodiment, relative condition of activity such as whether biometric data of one patient is located near the median value or is located near the end of a quartile is known, and thus, whether activity and the like more than assumed for a patient of each rank has been performed can be known.

### Second Embodiment

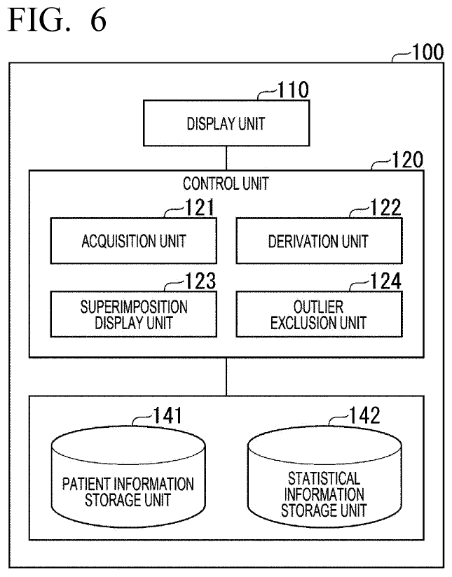

In addition to the configuration of the first embodiment, a second embodiment has a configuration including an outlier exclusion unit that excludes biometric statistical information using a threshold according to a distribution of the biometric statistical information. FIG. 6 is a functional block diagram illustrating a functional configuration of a condition analysis device 100 according to the second embodiment. In the functional configuration of the condition analysis device 100 according to the second embodiment, the same functional configuration as that of the condition analysis device 100 according to the first embodiment is denoted by the same reference numeral.

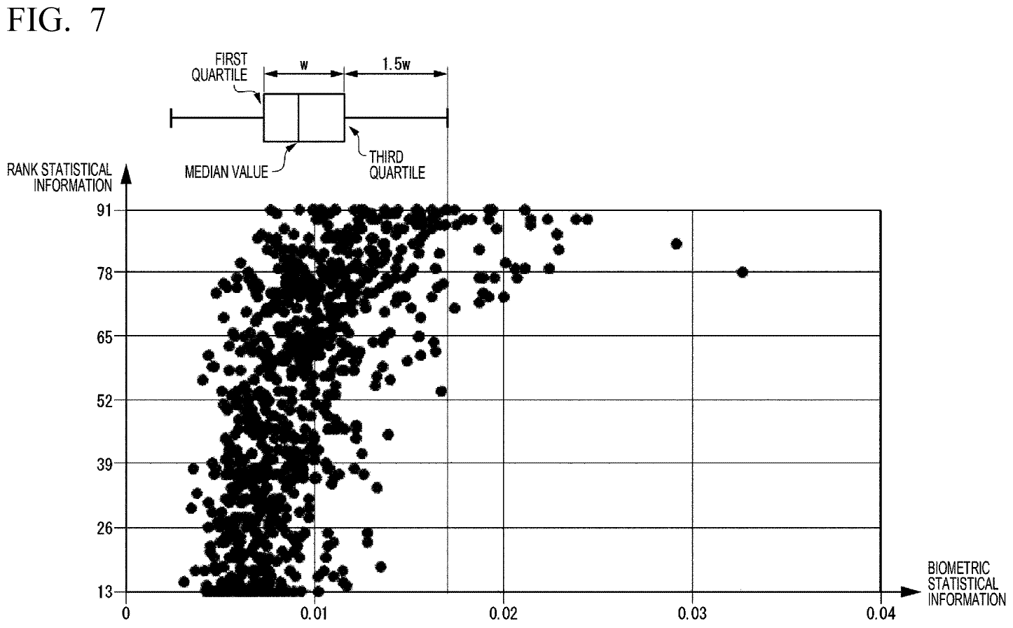

The outlier exclusion unit 124 of a control unit 120 of the condition analysis device 100 excludes biometric statistical information using a threshold according to a distribution of the biometric statistical information. For example, as described in Non Patent Literature 2, the threshold is obtained using quartile values (first quartile, median value, third quartile) of biometric data, and biometric statistical information deviating from the threshold is excluded. FIG. 7 is a diagram illustrating an example in which quartile value are used as thresholds according to a distribution of biometric statistical information. The vertical axis of the graph illustrated in FIG. 7 indicates rank statistical information, and the horizontal axis indicates the range in which the biometric statistical information is distributed. Note that the rank statistical information illustrated in FIG. 7 displays the total score (91 points) of FIM exercise items (13 items).

As illustrated in FIG. 7, the outlier exclusion unit 124 derives a first quartile, a median value, and a third quartile in the distribution of the biometric statistical information. Then, a threshold is determined using the width w between the first quartile and the third quartile. As an example of the threshold, a value obtained by adding 1.5 w obtained by multiplying the width w by 1.5 to the third quartile is set as the threshold. The outlier exclusion unit 124 excludes biometric statistical information that does not exist in a range from below the threshold to 0.

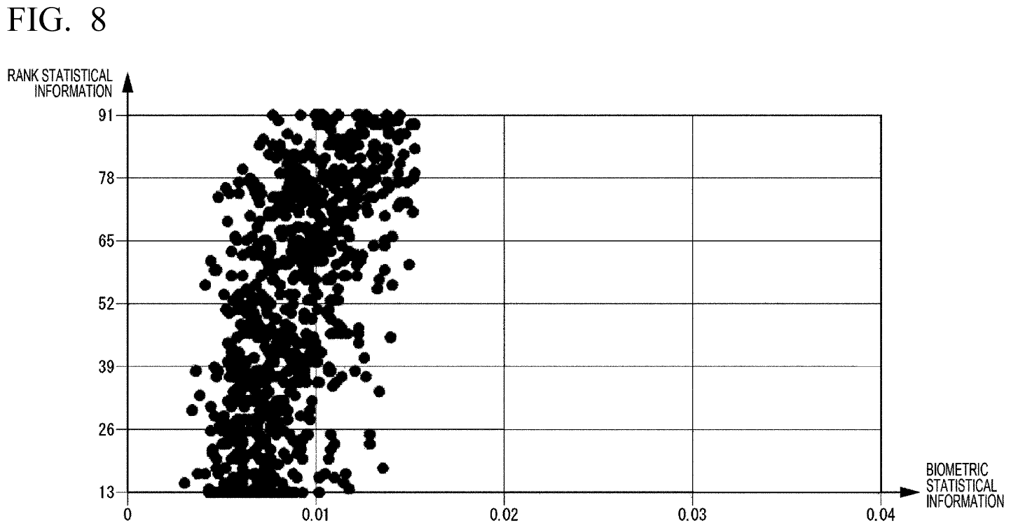

FIG. 8 is a diagram illustrating a graph after exclusion. FIG. 8 illustrates that the biometric statistical information that does not exist in the above range is excluded. In the present embodiment, outliers are excluded by a method using quartile values, but another method other than quartile values may be used as long as outliers can be excluded by the another method. Since the derivation unit 122 derives range information using biometric statistical information after exclusion, variation is reduced and influence on analysis can be reduced.

### Third Embodiment

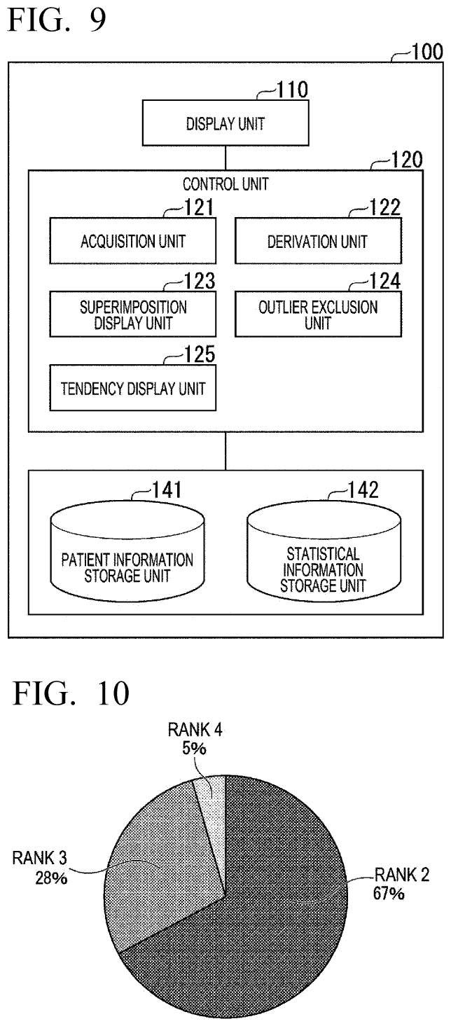

A third embodiment has a configuration including a tendency display unit 125 in addition to the configuration of the second embodiment. FIG. 9 is a functional block diagram illustrating a functional configuration of a condition analysis device 100 according to the third embodiment. In the functional configuration of the condition analysis device 100 according to the third embodiment, the same functional configuration as that of the condition analysis device 100 according to the second embodiment is denoted by the same reference numeral.

A control unit 120 according to the third embodiment includes the tendency display unit 125. The tendency display unit 125 detects one or more similar patients having rank statistical information and biometric statistical information similar to rank statistical information and biometric statistical information of one patient in a past period of time. The tendency display unit 125 acquires rank statistical information after a lapse of a predetermined period from the period of time of the detected similar patients, and displays a tendency of a rank indicated by the acquired rank statistical information.

The tendency display unit 125 first acquires rank statistical information and biometric statistical information of one patient. Then, the tendency display unit 125 detects one or more similar patients having rank statistical information and biometric statistical information similar to the rank statistical information and the biometric statistical information in a past period of time in a statistical database. Here, the rank statistical information “similar” to the rank statistical information of the one patient is rank statistical information indicating a value in the vicinity of a value indicated by the rank statistical information of the one patient. Similarly, the biometric statistical information “similar” to the biometric statistical information of the one patient is biometric statistical information indicating a value in the vicinity of a value indicated by the biometric statistical information of the one patient.

For example, when a value indicated by the rank statistical information of the one patient is a and b is a positive number, the vicinity is a section [a−b, a+b]. The value of b may be settable by an operator or may be set by default. Similarly, for example, when a value indicated by the biometric statistical information of the one patient is c and d is a positive number, the vicinity in the biometric statistical information is a section [c−d, c+d]. The value of d may be settable by an operator or may be set by default.

As illustrated in FIG. 3, the statistical database includes a date. Therefore, the tendency display unit 125 detects one or more similar patients having rank statistical information and biometric statistical information in a past period of time (for example, predetermined period before (for example, two weeks before) the present). Next, the tendency display unit 125 acquires rank statistical information after a lapse of a predetermined period from the period of time (for example, at present) of the detected similar patients. As a result, for example, 100 similar patients are detected, and current rank statistical information of the similar patients is acquired, so that the tendency display unit 125 can obtain a tendency (for example, proportion) of ranks of the similar patients after the predetermined period.

FIG. 10 is a diagram illustrating a display example by the tendency display unit 125. FIG. 10 is a pie chart illustrating the proportion of the ranks of the similar patients after the predetermined period. In the example of FIG. 10, since a rank 2 is 67%, a rank 3 is 28%, and a rank 4 is 5%, it can be seen that the rank of the one patient tends to be the rank 2. In this way, whether the one patient is in a group in which the condition of the one patient tends to rise or a group in which the condition of the one patient tends to stagnate can be determined.

### Fourth Embodiment

A fourth embodiment has a configuration including a prediction unit 126 in addition to the configuration of the third embodiment. The prediction unit 126 predicts rank statistical information and biometric statistical information after a lapse of a predetermined period from rank statistical information and biometric statistical information by performing learning using rank statistical information and biometric statistical information in a past period of time and rank statistical information and biometric statistical information after a lapse of a predetermined period from the past period of time.

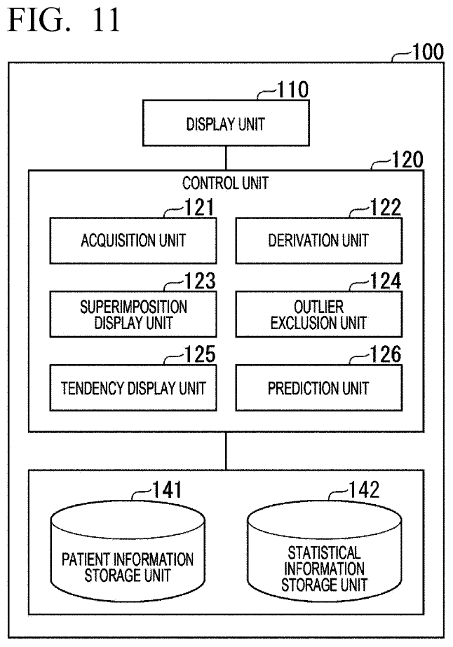

FIG. 11 is a functional block diagram illustrating a functional configuration of a condition analysis device 100 according to the fourth embodiment. In the functional configuration of the condition analysis device 100 according to the fourth embodiment, the same functional configuration as that of the condition analysis device 100 according to the third embodiment is denoted by the same reference numeral.

A control unit 120 according to the fourth embodiment includes the prediction unit 126. The prediction unit 126 includes a learning device. For example, random forest, gradient boosting, or the like may be used as the learning device. Biometric statistical information of another patient may be used as an explanatory variable, and rank statistical information of another patient acquired after the acquisition date of the biometric statistical information may be used as an objective variable. For example, if learning is performed using rank statistical information two weeks after the time point at which biometric statistical information is acquired as an objective variable, a learning model for predicting rank statistical information after about two weeks from the biometric statistical information (hereinafter, the learning model is referred to as a “learning model A”) can be acquired.

When a new learning model (hereinafter, the learning model is referred to as a “learning model B”) is acquired by changing the objective variable to rank statistical information after four weeks and by newly performing learning, rank statistical information after two weeks from a measurement time point of biometric statistical information can be predicted from the learning model A, using the biometric statistical information of any patient. Furthermore, rank statistical information after four weeks can be predicted from the learning model B.

Note that biometric statistical information can be predicted from rank statistical information by performing learning using rank statistical information as an explanatory variable and using biometric statistical information as an objective variable.

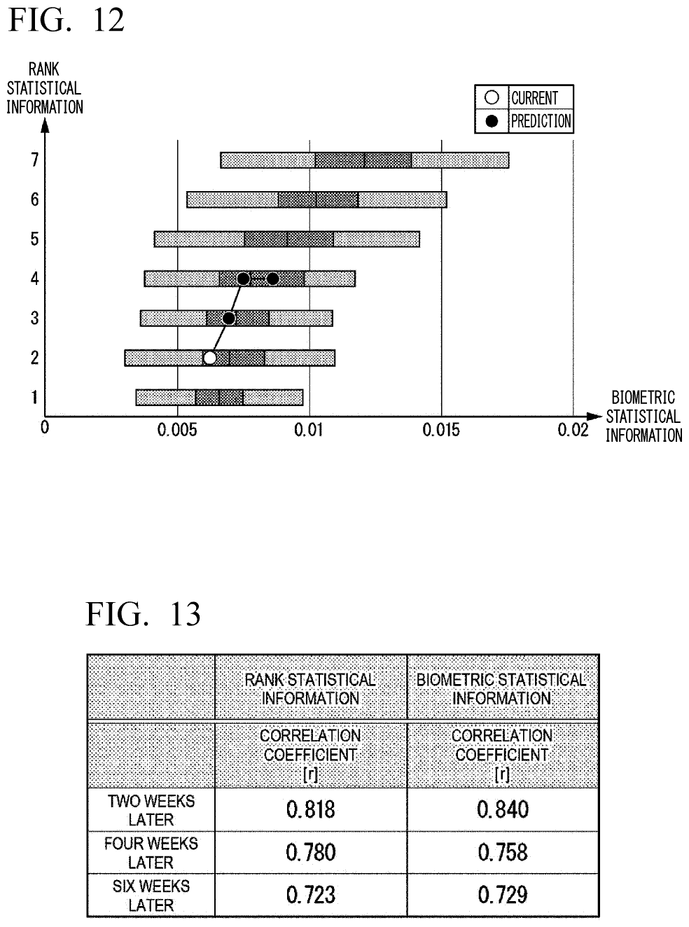

Images indicating predicted rank statistical information and biometric statistical information after two weeks and four weeks may be superimposed and displayed on a graph. FIG. 12 is a diagram illustrating an example of a graph on which predicted rank statistical information and biometric statistical information are superimposed. The vertical axis of the graph illustrated in FIG. 12 indicates rank statistical information, and the horizontal axis indicates the range in which the biometric statistical information is distributed. A white circle indicates current rank statistical information and biometric statistical information of the patient, and black circles indicate predicted rank statistical information and biometric statistical information. FIG. 12 illustrates, as an example, results of prediction after two weeks, four weeks, and six weeks. In a case of FIG. 12, it is predicted that both the rank statistical information and the biometric statistical information will increase from the present.

FIG. 13 is a diagram illustrating correlation coefficients of predicted values. FIG. 13 illustrates correlation coefficients after two weeks, four weeks, and six weeks for each of rank statistical information and biometric statistical information. About 0.8 is recorded as a correlation coefficient of rank statistical information after two weeks, and about 0.72 is recorded as a correlation coefficient after six weeks, which is the farthest from the time of acquisition of the biometric statistical information, indicating high reliability. Therefore, the prediction unit 126 can predict condition of one patient up to six weeks later with high reliability.

As described above, according to the present embodiment, by making it possible to predict rank statistical information and biometric statistical information, suitable assistance regarding rehabilitation can be provided, such as assistance of creation of a rehabilitation treatment plan and contribution to improvement of motivation of a patient for rehabilitation training.

### Fifth Embodiment

A fifth embodiment has a configuration including a complement unit 127 in addition to the configuration of the fourth embodiment. The complement unit 127 complements biometric statistical information from rank statistical information or complements rank statistical information from biometric statistical information by learning rank statistical information and biometric statistical information for each patient.

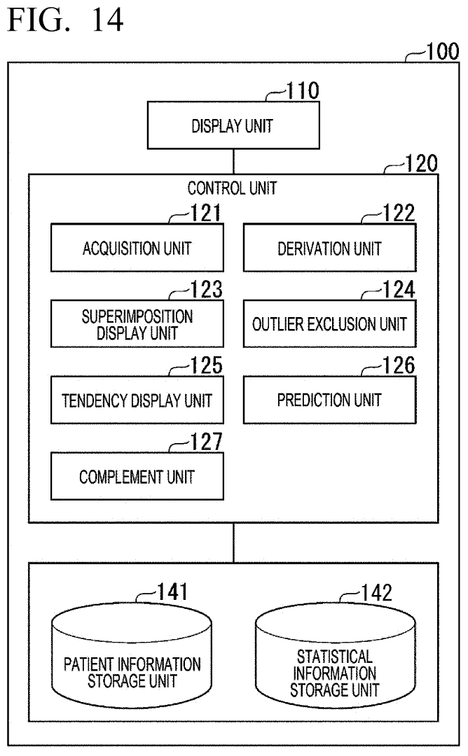

FIG. 14 is a functional block diagram illustrating a functional configuration of a condition analysis device 100 according to the fifth embodiment. In the functional configuration of the condition analysis device 100 according to the fifth embodiment, the same functional configuration as that of the condition analysis device 100 according to the fourth embodiment is denoted by the same reference numeral.

A control unit 120 according to the fifth embodiment includes the complement unit 127. In a case where there is absence such as nonexistence of any one or both of rank statistical information and biometric data of a patient, the complement unit 127 complements the absent information using a learning device. As a method of complementing absent information in the complement unit 127, for example, as in Reference Literature 3, a single substitution method or a multiple substitution method may be used.

In a case where rank statistical information is absent, the complement unit 127 may complement the rank statistical information. Such complementation can be performed using the method of Reference Literature 3 using rank statistical information as an objective variable and biometric statistical information as an explanatory variable.

Similarly, in a case where biometric statistical information is absent, the complement unit 127 may complement the biometric statistical information. In this case, the complement unit 127 may perform learning using one variable (any one of heart rate, body motion, exercise intensity, and the like) of the biometric statistical information as an objective variable and other biometric statistical information and rank statistical information as explanatory variables, and repeatedly perform complementation by changing the objective variable such that all the absence of the biometric statistical information is complemented.

Even in a case where both biometric statistical information and rank statistical information are absent, the complement unit 127 can complement all the absent information by sequentially complementing them. In a case where it is considered more appropriate to leave the absent information in the rank statistical information or the biometric statistical information as absent, only specific biometric statistical information or rank statistical information may be complemented.

In the present embodiment, a rank of each item of FIM used in rank statistical information may not be input by an operator for some reason, and in this case, the rank statistical information is absent information. In a case where data from a wearable device is used as biometric statistical information, there is a case where data cannot be acquired due to non-wearing of the wearable device or a measurement error, and in this case, biometric statistical information is absent information. Even if absent information is generated due to such circumstances, the absent information can be complemented, and eventually, highly reliable analysis of a condition can be provided, according to the present embodiment.

Although the embodiments of the present invention have been described in detail with reference to the drawings, specific configurations are not limited to the embodiments and include design and the like within the gist of the present invention.

### INDUSTRIAL APPLICABILITY

The present invention is applicable to analysis and diagnosis of rehabilitation.

### REFERENCE SIGNS LIST

100 Condition analysis device
110 Display unit
120 Control unit
121 Acquisition unit
122 Derivation unit
123 Superimposition display unit
124 Outlier exclusion unit
125 Tendency display unit
126 Prediction unit
127 Complement unit
141 Patient information storage unit
142 Statistical information storage unit

1. A condition analysis device comprising:
an acquisition circuitry that acquires rank information obtained by ranking a degree of self-support or magnitude of disorder of a patient and biometric information of the patient;
a derivation circuitry that derives, for each rank, range information indicating a range in which the biometric information is distributed; and
a superimposition display circuitry that displays the range information as an image for each rank, and superimposes and displays a predetermined image on the image at a position corresponding to the biometric information of one patient in a range indicated by the image.

2. The condition analysis device according to claim 1 comprising an outlier exclusion circuitry that excludes biometric information using a threshold according to a distribution of the biometric information.

3. The condition analysis device according to claim 1 comprising a tendency display circuitry that detects one or more similar patients having the rank information and the biometric information similar to the rank information and the biometric information of the one patient in a period of time in a past, acquires the rank information of detected one or more similar patients after a lapse of a predetermined period from the period of time, and displays a tendency of a rank indicated by the acquired rank information.

4. The condition analysis device according to claim 1 comprising a prediction circuitry that predicts the rank information and the biometric information after a lapse of a predetermined period from the period of time using the rank information and the biometric information in a period of time in a past and the rank information and the biometric information after the lapse of the predetermined period.

5. The condition analysis device according to claim 1 comprising a complement circuitry that complements the biometric information from the rank information or complements the rank information from the biometric information.

6. The condition analysis device according to claim 1,
wherein the rank information is information determined on a basis of functional independence measure (FIM) or stroke impairment assessment set (SIAS), and the biometric information is information determined on a basis of a heart rate or body motion.

7. A condition analysis method comprising:
acquiring rank information obtained by ranking a degree of self-support or magnitude of disorder of a patient and biometric information of the patient;
deriving, for each rank, range information indicating a range in which the biometric information is distributed; and
displaying the range information as an image for each rank, and superimposing and displaying a predetermined image on the image at a position corresponding to the biometric information of one patient in a range indicated by the image.
